(19) United States
(12) Patent Application Publication
(10) Pub. No.: US 2004/0059390 A1
(43) Pub. Date: Mar. 25, 2004

(54) Device wtih redetection therapy thereshold

(75) Inventors: Tran Thong, Portland, OR (US); Mrigank Shekhar, Vancouver, WA (US); Indra B. Nigam, Tigard, OR (US)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co. Ingenieurbuero Berlin

(21) Appl. No.: 10/666,752

(22) Filed: Sep. 18, 2003

Related U.S. Application Data
(60) Provisional application No. 60/411,905, filed on Sep. 19, 2002.

(51) Int. Cl. A61N 1/39

(52) U.S. Cl. 607/5; 607/14

(57) ABSTRACT

An arrangement for treating rhythm disturbances of a heart (H), especially tachycardia and fibrillation, has a device (FD) for detecting the heart rhythm and determining when a fibrillation threshold limit is exceeded and a therapy device or stimulator (IG). The therapy device (IG) is connected to the heart rhythm detecting device (FD), to begin to treat the fibrillation when the fibrillation threshold limit is exceeded. The heart rhythm detecting device determines whether a redetection threshold limit is still exceeded after the therapy device has treated the fibrillation, the redetection threshold limit being lower than the fibrillation threshold limit and higher than a tachycardia threshold limit. The therapy device continues to treat the fibrillation as long as the heart rhythm detector determines that the redection threshold limit is exceeded.

This application claims the benefit of U.S. Provisional Application Serial No. 60/411,905 which is hereby incorporated herein by reference.

### TECHNICAL FIELD

The present invention relates to an device for treating cardiac arrthythmias and fibrillation. Particularly, the device relates to an implantable cardioverter/defibrillator having the capacity to provide redetection therapy.

### BACKGROUND OF THE ART

Cardiac tachycardia is heart rate, high compared to the normal rate, that provides inadequate pumping when compared to a normal healthy heart rhythm. Cardiac fibrillation is an even higher heart rate, where no coordinated heart contraction occurs, rendering the situation life-threatening.

Both tachycardia and fibrillation are known to be treatable with implantable cardioverter/defibrillators (“ICD”). These devices are connected to intercardiac electrodes that sense electrical signals in the inner heart. When fed to a rate detector in the ICD, the electrical signals provide a type of intracardial electrocardiogram (“EKG”) that represents heart activity. Particularly, the rate detector in an ICD can focus on the so-called QRS complexes of an EKG. The QRS complexes represent the portion of the heartbeat in which depolarization of the ventricular tissue occurs and the ventricle contracts. When a pre-determined rate threshold is exceeded, the rate detector determines the heart to be in tachycardia. This is referred to as the tachycardia threshold. Beyond the tachycardia threshold is a higher threshold, also predetermined, above which the heart is determined to be in fibrillation. This is the fibrillation threshold.

Rate detectors in ICDs of the prior art are equipped to detect tachycardia and fibrillation. Once either is detected, the prior art ICD will initiate treatment by supplying an antitachycardiac pacing sequence or a defibrillation electrical impulse to the heart tissue, using the intracardial electrodes.

In the prior art ICDs, the device is arranged to determine if a subsequent fibrillation occurs within a predetermined time after a first fibrillation. If the second fibrillation occurs within the time parameter, the subsequent fibrillation will be treated more aggressively than the first. By “more aggressively,” we mean that the defibrillation electrical impulse is of a higher energy. If the detector does not detect a heart rate continuing above the fibrillation threshold, further defibrillation does not occur. However, if the heart rate is below the fibrillation threshold but above the tachycardia threshold, the ICD switches to providing antitachycardia pacing to the heart tissue.

Antitachycardia pacing, while necessary when successful, may pose significant deleterious effects to the patient when not successful. First, it may cause the heart rate to increase above the fibrillation threshold, causing the ICD to deliver the more aggressive second defibrillation electrical impulse described above. Unsuccessful antitachycardia pacing and defibrillation may alternate over several cycles, during which more aggressive defibrillation electrical impulses are delivered by the ICD.

Another therapy method known in the art is to continue defibrillation therapy, once it is started, until the heart rate falls below the tachycardia threshold, rather than stopping the defibrillation therapy after the heart rate falls below the fibrillation threshold. The disadvantageous result of this is that the patient only exhibiting tachycardia and not exhibiting fibrillation is receiving the more disruptive defibrillation electrical impulses.

It is known that the application of defibrillation therapy to the heart may induce the medical condition of shock in the patient, a symptom of which is increasing heart rate. This shock is caused by the psychological stress of the defibrillation electrical impulses. The higher heart rate caused by the medical condition of shock in the patient is not pathological, and should not be treated by the ICD. However, the ICDs of the prior art, and particularly the ICDs of the type where defibrillation therapy is continued until the heart rate is below the tachycardia threshold, would continue the defibrillation therapy during this stress-induced medical condition of shock.

It is therefore, an object of the present invention to avoid the disadvantages of the prior art ICD defibrillation therapies by stopping the defibrillation therapy once the heart rate or frequency has slowed below a therapy redetection threshold limit. Such a therapy redetection threshold limit would lie above the tachycardia threshold limit, but below the fibrillation threshold limit.

### SUMMARY OF THE INVENTION

This object is achieved by an arrangement for treatment of rhythm disturbances, especially tachycardia and fibrillation, of a heart. Such an arrangement comprises a device for detecting the heart rhythm and determining when a fibrillation threshold limit is exceeded and a therapy device. The therapy device is connected to the heart rhythm detecting device, to begin to treat the fibrillation when the fibrillation threshold limit is exceeded. In the inventive arrangement, the heart rhythm detecting device determines whether a redetection threshold limit is still exceeded after the therapy device has treated the fibrillation, the redetection threshold limit being lower than the fibrillation threshold limit and higher than a tachycardia threshold limit. The therapy device continues to treat the fibrillation as long as the heart rhythm detector determines that the redection threshold limit is exceeded.

In one embodiment, the therapy device delivers a series of electrical impulses to the heart. In such a device the heart rhythm detector detects either an atrial or ventricular fibrillation and the therapy device treats that particular fibrillation.

In the same embodiment, the heart rhythm detector determines when a tachycardia is occurring, and the therapy device begins to treat the tachycardia when the tachycardia is detected. In such an embodiment, the therapy device is designed so that no tachycardia treatment is performed during a fibrillation treatment. In this embodiment, the heart rhythm detector is designed so that the redetection threshold limit is ignored, when a fibrillation is determined after either a fibrillation or a tachycardia treatment is started. The therapy device treats tachycardia through overdriving at a stimulation frequency and the therapy device is designed so that the overdriving stimulation frequency is in the range of 10 to 50 beats/minute higher than the tachycardia frequency.

### BRIEF DESCRIPTION OF THE DRAWINGS

The present invention will be better understood when reference is made to the accompanying drawings, wherein identical parts are identified with identical reference numerals and wherein:

FIG. 1 shows an implantable device incorporating the arrangement for treatment of heart rhythm disturbances, as described in the present invention;

FIG. 2 shows an arrangement for treatment of heart rhythm disturbances according to the working examples; and

FIG. 3 schematically shows the operational principles of the arrangement for the treatment of heart rhythm disturbances

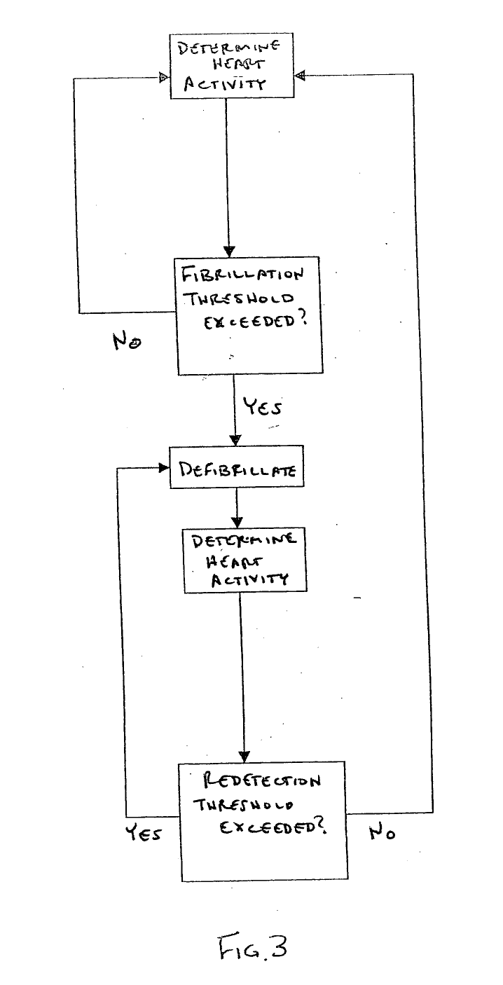

### DETAILED DESCRIPTION OF A PREFERRED EMBODIMENT

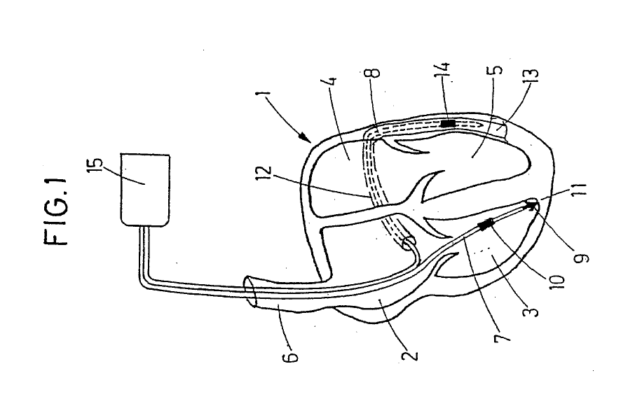

Aspects of the present invention will be better understood when reference is first made to FIG. 1, which shows only aspects of the invention previously known in the prior art. The figure itself is taken from U.S. Pat. No. 6,339,724 B1, issued to Thong on 15 Jan. 15, 2002 and assigned to the present applicant. FIG. 1 shows a diagrammatic section of a heart 1, comprising a right atrium 2, right ventricle 3, left atrium 4 and left ventricle 5. Two catheters 7, 8 are led via the vena cava 6 into the heart, catheter 7 being guided into the right atrium and ventricle and catheter 8 being advanced via the coronary sinus 12 as far as the ensuing great cardiac vein 13. Catheter 7 has a tip electrode 9 anchored at the pointed end of the right ventricle 3 in the myocardium 11. A ring electrode 10 of catheter 7 floats freely in the blood stream within right ventricle 3. Catheter 8 has an electrode 14 along its length. Cardiac pacemaker 15 is conventionally implanted subcutaneously and is connected to electrodes 9, 10, 14 via corresponding inputs.

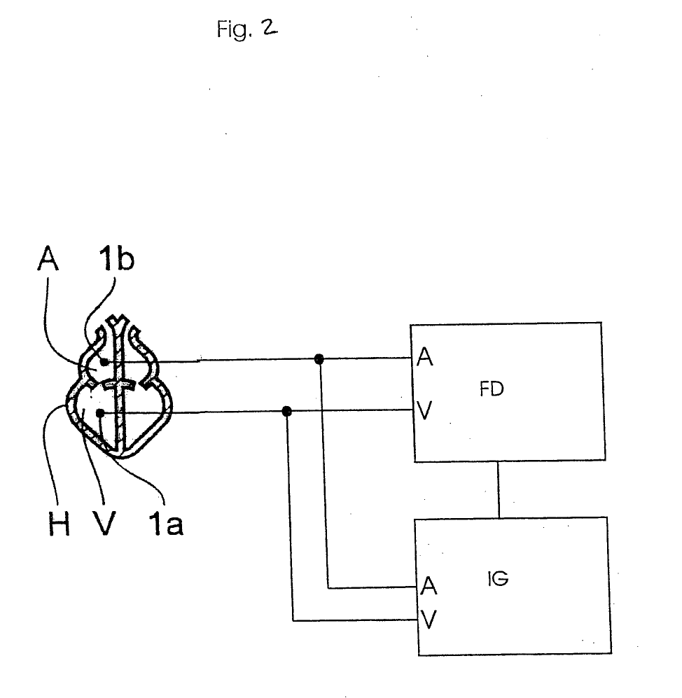

FIG. 2 schematically shows a fibrillation detector FD and a stimulator IG which could be positioned inside a pacemaker, such as pacemaker 15 of FIG. 1. The fibrillation detector FD has two inputs A1, V1, in which electrodes 1a, 1b are connected, respectively. Electrode 1b is located in an atrium A of a human heart H, which is shown schematically. Electrode 1a is located in a ventricle V of the heart and connected to the fibrillation detector through input V1. The fibrillation detector receives electrical signals through electrodes 1a and 1b. These signals represent the activity of the atrium A and the ventricle V. The stimulator IG is likewise connected with the electrode 1b in atrium A through output A2 and with the electrode 1a in ventricle B through output V2. Accordingly, the stimulator is able to deliver electrical impulses through the respective outputs A2 and V2 to the atrium A and the ventricle V through electrodes 1b and 1a. Also shown is a lead L connecting the stimulator IG to the fibrillation detector. The lead L serves the function of transmitting directions or orders between the fibrillation detector and stimulator, as well as transmitting a response that the signal has been received. The stimulator IG or the fibrillation detector FD communicate with each other through the lead L.

FIG. 2 shows, in a block diagram, the principal functionality of the arrangement illustrated in FIG. 1. In step 1, the cardiac activity is determined. In this, the fibrillation detector FD captures and records the electrical activity in atrium A and ventricle V, by way of the electrodes 1a and 1b and the inputs A1, V1. The recordation occurs over a time period that is longer than the period or cycle of a normally beating heart. In the next step 2, it is determined whether a fibrilllation threshold value has been exceeded. To do that, the recorded atrial and ventricular cardiac activity is compared and analyzed against predetermined parameters. The frequency of the captured signals is included in any case in the measurement of fibrilllation. When the frequency of captured signals at A1 and V1 is less than the fibrillation threshold, the procedure returns to step 1, in which the cardiac activity is again determined. This is shown schematically in FIG. 2 as the “No” path leading away from the step 2 to step 1.

If, on the other hand, the determination in step 2 is that the fibrillation threshold as detected by electrode 1b has been exceeded, then atrial fibrillation is occurring in the heart H. In that case, the “Yes” path from step 2 to step 3 is followed. The stimulator IG receives an order from the fibrillation detector FD over the lead L to dispatch a defibrillation stimulation to the atrium A of the heart H. The order is obeyed by the stimulator IG sending a succession or series of electrical impulses from output A2 through lead L to electrode 1b. After the defibrillation sequence, the stimulator IG sends an order to the fibrillation detector to reinstitute determination of cardiac activity, so that the effectiveness of the defibrillation sequence can be determined. This is represented schematically by the single exit pathway from the box representing step 3, that is, the pathway that connects step 3 to step 4.

The activity in step 4, that is, the step following a defibrillation sequence, is really the same procedure as is followed in step 1. To be specific, the fibrillation detector FD again captures and records the electrical activity in atrium A and ventricle V, by way of the electrodes 1a and 1b and the inputs A1, V1. The recordation occurs over a time period that is longer than the period or cycle of a normally beating heart. However, the fact that a defibrillation sequence has already occurred means that the information obtained is used to determine whether a redetection threshold value has been exceeded, rather than a fibrillation threshold limit To do that, the frequency of electrical signals from the atrium are compared against a predetermined redetection threshold limit. The redetection threshold limit is lower than the fibrillation threshold limit. When the comparison of the cardiac activity shows that the redetection threshold limit is exceeded, then the “Yes” path leading away from step 5 is followed to step 3, and the stimulator IG commences a further defibrillation sequence, with the path to step 4 being followed after the end of the sequence. This sequence from step 3 through step 4 and to step 5 continues until the cardiac activity is found to be below the redetection threshold limit. When this occurs at last, then the pathway labeled “No” for the question “Redetection threshold limit exceeded?” is followed, and fibrillation treatment by the stimulator IG is stopped. The system has then returned to step 1 and the procedure may be repeated as necessary.

The detection and treatment of a ventricular fibrillation is accomplished in an analogous manner as the detection and treatment of an atrial fibrillation as described above, except that the signal from electrode 1a located in the ventricle is used in step 2 (instead of electrode 1b in the atrium) to determine that a defibrillation signal needs to be sent to the ventricle. Also, instead of sending the defibrillation sequence of impulses through the lead L to electrode 1b, as in the case of atrial fibrillation, the signal is sent to electrode 1a.

What is claimed is:

1. An arrangement for treatment of rhythm disturbances, especially tachycardia and fibrillation, of a heart, comprising:
a device for detecting the heart rhythm and determining when a fibrillation threshold limit is exceeded; and
a therapy device, connected to the heart rhythm detecting device, to begin to treat the fibrillation when the fibrillation threshold limit is exceeded;
wherein the heart rhythm detecting device determines whether a redetection threshold limit is still exceeded after the therapy device has treated the fibrillation, the redetection threshold limit being lower than the fibrillation threshold limit and higher than a tachycardia threshold limit, and
wherein the therapy device continues to treat the fibrillation as long as the heart rhythm detector determines that the redection threshold limit is exceeded.

2. The arrangement of claim 1, wherein the therapy device delivers a series of electrical impulses to the heart.

3. The arrangement of claim 1, wherein
the heart rhythm detector detects an atrial fibrillation and
the therapy device treats the atrial fibrillation.

4. The arrangement of claim 2, wherein
the heart rhythm detector detects an atrial fibrillation and
the therapy device treats the atrial fibrillation.

5. The arrangement of claim 2, wherein
the heart rhythm detector comprises an electrode that is situated in a region of an atrium of the heart to detect the electrical activity thereof, and
the therapy device is connected to the atrial electrode to deliver electrical impulses to the atrium.

6. The arrangement of claim 1, wherein
the heart rhythm detector detects a ventricular fibrillation and
the therapy device treats the ventricular fibrillation.

7. The arrangement of claim 2, wherein
the heart rhythm detector detects a ventricular fibrillation and
the therapy device treats the ventricular fibrillation.

8. The arrangement of claim 3, wherein
the heart rhythm detector detects a ventricular fibrillation and
the therapy device treats the ventricular fibrillation.

9. The arrangement of claim 4, wherein
the heart rhythm detector detects a ventricular fibrillation and
the therapy device treats the ventricular fibrillation.

10. The arrangement of claim 5, wherein
the heart rhythm detector detects a ventricular fibrillation and
the therapy device treats the ventricular fibrillation.

11. The arrangement of claim 6, wherein
the heart rhythm detector comprises an electrode that is situated in a region of a ventricle of the heart to detect the electrical activity thereof, and
the therapy device is connected to the atrial electrode to deliver electrical impulses to the ventricle

12. The arrangement of claim 7, wherein
the heart rhythm detector comprises an electrode that is situated in a region of a ventricle of the heart to detect the electrical activity thereof, and
the therapy device is connected to the atrial electrode to deliver electrical impulses to the ventricle

13. The arrangement of claim 8, wherein
the heart rhythm detector comprises an electrode that is situated in a region of a ventricle of the heart to detect the electrical activity thereof, and
the therapy device is connected to the atrial electrode to deliver electrical impulses to the ventricle

14. The arrangement of claim 9, wherein
the heart rhythm detector comprises an electrode that is situated in a region of a ventricle of the heart to detect the electrical activity thereof, and
the therapy device is connected to the atrial electrode to deliver electrical impulses to the ventricle

15. The arrangement of claim 10, wherein
the heart rhythm detector comprises an electrode that is situated in a region of a ventricle of the heart to detect the electrical activity thereof, and
the therapy device is connected to the atrial electrode to deliver electrical impulses to the ventricle

16. The arrangement of claim 1, wherein the heart rhythm detector further comprises:
a means to determine a heart activity limit and detect whether the heart activity exceeds the fibrillation threshold limit or the redetection threshold limit.

17. The arrangement of claim 2, wherein the heart rhythm detector further comprises:
a means to determine a heart activity limit and detect whether the heart activity exceeds the fibrillation threshold limit or the redetection threshold limit.

18. The arrangement of claim 11, wherein the heart rhythm detector further comprises:
a means to determine a heart activity limit and detect whether the heart activity exceeds the fibrillation threshold limit or the redetection threshold limit.

19. The arrangement of claim 12, wherein the heart rhythm detector further comprises:
a means to determine a heart activity limit and detect whether the heart activity exceeds the fibrillation threshold limit or the redetection threshold limit.

20. The arrangement of claim 13, wherein the heart rhythm detector further comprises:
a means to determine a heart activity limit and detect whether the heart activity exceeds the fibrillation threshold limit or the redetection threshold limit.

21. The arrangement of claim 14, wherein the heart rhythm detector further comprises:
a means to determine a heart activity limit and detect whether the heart activity exceeds the fibrillation threshold limit or the redetection threshold limit.

22. The arrangement of claim 15, wherein the heart rhythm detector further comprises:
a means to determine a heart activity limit and detect whether the heart activity exceeds the fibrillation threshold limit or the redetection threshold limit.

23. The arrangement of claim 16, wherein the heart rhythm detector further comprises:
the heart activity limit-determining means uses at least one of a plurality of heart activity parameters to determine the heart activity limit.

24. The arrangement of claim 23, wherein the heart activity parameter used is heart rate.

25. The arrangement of claim 23, wherein the heart activity parameter used is instantaneous rate of change in heart rate.

26. The arrangement of claim 23, wherein the heart activity parameter used is the difference between auricular activity and heart chamber activity.

27. The arrangement of claim 1, wherein:
the heart rhythm detector determines when a tachycardia is occurring, and
the therapy device begins to treat the tachycardia when the tachycardia is detected.

28. The arrangement of claim 4, wherein:
the heart rhythm detector determines when a tachycardia is occurring, and
the therapy device begins to treat the tachycardia when the tachycardia is detected.

29. The arrangement of claim 27, wherein
the heart rhythm detector determines that a tachycardia is occurring when the tachycardia threshold limit is exceeded, when the fibrillation threshold rate is exceeded and also when the redetection threshold limit is exceeded.

30. The arrangement of claim 28, wherein
the heart rhythm detector determines that a tachycardia is occurring when the tachycardia threshold limit is exceeded, when the fibrillation threshold rate is exceeded and also when the redetection threshold limit is exceeded.

31. The arrangement of claim 27, wherein:
the therapy device is designed so that no tachycardia treatment is performed during a fibrillation treatment.

32. The arrangement of claim 28, wherein:
the therapy device is designed so that no tachycardia treatment is performed during a fibrillation treatment.

33. The arrangement of claim 31, wherein
the heart rhythm detector is designed so that the redetection threshold limit is ignored, when a fibrillation is determined after either a fibrillation or a tachycardia treatment is started.

34. The arrangement of claim 32, wherein
the heart rhythm detector is designed so that the redetection threshold limit is ignored, when a fibrillation is determined after either a fibrillation or a tachycardia treatment is started.

35. The arrangement of claim 33, wherein
the therapy device treats an observed tachycardia through overdriving at a stimulation frequency.

36. The arrangement of claim 34, wherein
the therapy device treats an observed tachycardia through overdriving at a stimulation frequency.

37. The arrangement of claim 35, wherein
the therapy device is designed so that the overdriving stimulation frequency is in the range of 10 to 50 beats/minute higher than a frequency of the observed tachycardia.

38. The arrangement of claim 36, wherein
the therapy device is designed so that the overdriving stimulation frequency is in the range of 10 to 50 beats/minute higher than a frequency of the observed tachycardia.

39. The arrangement of claim 35, wherein
the therapy device is designed so that the overdriving stimulation frequency is increased in the absence of a successful treatment.

40. The arrangement of claim 36, wherein
the therapy device is designed so that the overdriving stimulation frequency is increased in the absence of a successful treatment.

41. The arrangement of claim 37, wherein
the therapy device is designed so that the overdriving stimulation frequency is increased in the absence of a successful treatment.

42. The arrangement of claim 38, wherein
the therapy device is designed so that the overdriving stimulation frequency is increased in the absence of a successful treatment.
